(19) United States
(12) Patent Application Publication
(10) Pub. No.: US 2004/0077610 A1
(43) Pub. Date: Apr. 22, 2004

(54) Medicament containing a polyamine as an active substance

(76) Inventors: Tobias Schlapp, Koln (DE); Sonja Maria Friederichs, Lindlar (DE); Martin Vollmer, Leverkusen (DE)

Correspondence Address: Jeffrey M Greenman, Bayer Corporation, 400 Morgan Lane, West Haven, CT, 06516, US

(21) Appl. No.: 10/250,511

(22) PCT Filed: Dec. 19, 2001
(86) PCT No.: PCT/EP01/15005

(30) Foreign Application Priority Data
Dec. 29, 2000 (DE) 100-65-710-9

(51) Int. Cl. A61K 31/56; A61K 31/17; A61K 31/13

(52) U.S. Cl. 514/169; 514/588; 514/674

(57) ABSTRACT

The present invention relates to medicaments which comprise a polyamine as the active substance and to the use of a polyamine for producing immunostimulatory medicaments or medicaments for the treatment and/or prophylaxis of various diseases in humans and animals.

The present invention relates to medicaments which comprise a polyamine as the active substance and to the use of a polyamine for producing immunostimulating medicaments and/or medicaments for the treatment and/or prophylaxis of various diseases in humans and animals.

For a relatively long time now, a product for inducing “paraspecific immunity”—what is termed an immunostimulator or paraimmunity inducer—has been employed therapeutically, metaphylactically and prophylactically in veterinary medical practice. As an example, immunostimulators can consist of chemically inactivated Parapoxvirus ovis, strain D 1701 (DE-A 35 04 940). BAYPAMUN® is a product which is prepared on the basis of this virus.

In the animal, the inactivated parapoxvirus induces nonspecific protection against infections caused by a very wide variety of pathogens. It is assumed that various mechanisms of the body's own defence system are responsible for mediating this protection.

These mechanisms include the induction of interferon, the activation of natural killer cells, the induction of “colony-stimulating activity” (CSA) and the stimulation of lymphocyte proliferation. Earlier investigations into the mechanism of action demonstrated that interleukin 2 and interferon α were stimulated (Steinmassl & Wolf, 1990).

In addition to this, immunostimulators, such as unmethylated, CpG-containing oligonucleotides (WO 98/18810), can be used for activating the nonadaptive immune system and for fortifying the body against the appearance of disease. pathogens. It has already been possible to show experimentally, in various mouse models, that a single administration can activate the initial immune response and prevent an infection with a variety of pathogens, for example infection with Listeria monocytogenes (Elkins et al., 1999; Krieg et al., 1998; Oxenius et al., 1999), Francisella tularensis (Elkins et al., 1999), Leishmania (Walker et al., 1999; Zimmeremann et al., 1998), anthrax, ebola and malaria (Krieg, 2000; Klinman et al., 1999).

In the analysis of the mechanism of action, it was possible to demonstrate that murine B cells, macrophages, dendritic cells and NK cells are all stimulated by the CpG-containing oligonucleotides. In addition, it was demonstrated that the cytokines IL-18, IL-12 and interferon γ were induced (Krieg, 2000).

The object of the present invention was to provide medicaments which comprise novel immunostimulators which, while exhibiting a similar activity to that of Parapox ovis, can be synthesized chemically and are therefore cheaper to produce and easier to combine with chemotherapeutic agents.

The object was achieved by providing medicaments which comprise a polyamine as the active substance.

The polyamines which are classified as being active substances contain at least 10 monomer units or at least 10 nitrogen atoms, preferably at least 45 monomer units or. at least 45 nitrogen atoms.

The polyamine can have a linear or branched structure.

The polyamine is preferably soluble or dispersible in water; a partial protonation, which is dependent on the pH, takes place in aqueous media. The degree of protonation can be determined by means of physicochemical measurement methods such as zeta potential measurements.

Preference is furthermore given to the polyamine possessing hydrophobic substituents.

The hydrophobic substituents can be arranged on the polymer either as side chains or terminally. The degree of substitution (the percentage of functionalized N atoms in the main polymer chain) is preferably between 0.01 and 10 per cent.

Suitable hydrophobic substituents are, in particular, alkyl chains, acyl chains or steroid-like substituents. Acyl chains are particularly suitable hydrophobic substituents. Hydrophobic substituents which can be introduced by adding the nitrogen function of the main polymer chain to isocyanates or to α,β-unsaturated carbonyl compounds are also suitable.

Particular preference is given to the polyamine being a polyethyleneimine.

A polyethyleneimine which can preferably be used for producing the medicament has the following general formula:

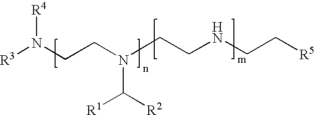

in which, in each individual [CH2—CH2—N] unit

R1 denotes hydrogen, methyl or ethyl, and

R2 denotes alkyl having from 1 to 23 carbon atoms, preferably alkyl having from 12 to 23 carbon atoms, particularly preferably alkyl having 17 carbon atoms

and in which

R3 and R4 (end groups) denote, independently of each other, hydrogen and alkyl having from 1 to 24 carbon atoms, preferably alkyl having from 13 to 24 carbon atoms, particularly preferably alkyl having 18 carbon atoms, or possess a structure which is dependent on the initiator,

with R5 (end group) being a substituent which is dependent on the termination reaction, for example hydroxyl, NH2, NHR or NR2, with it being possible for the R radicals to correspond to the end groups R3 and R4,

and with the average degree of polymerization P=(m+n) being in the range from 45 to 5250, preferably in the range from 250 to 2250, particularly preferably in the range from 500 to 2050, and n a×P with 0.0001<a<0.1, preferably 0.01<a<0.05, and particularly preferably a=0.03.

In this connection, the units m and n are not block structures but are instead distributed randomly in the polymer.

Another polyethyleneimine which can preferably be used for producing the medicament has the following general formula:

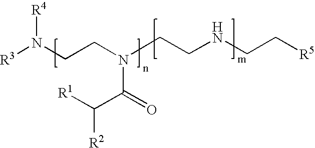

in which, in each individual [CH2—CH2—N] unit,

R1 denotes hydrogen, methyl or ethyl, and

R2 denotes alkyl having from 1 to 22 carbon atoms, preferably alkyl having from 11 to 22 carbon atoms, particularly preferably alkyl having 16 carbon atoms,

and in which

R3 and R4 (end groups) denote, independently of each other, hydrogen or acyl having from 1 to 24 carbon atoms, preferably acyl having from 13 to 24 carbon atoms, particularly preferably acyl having 18 carbon atoms, or possess a structure which is dependent on the initiator,

with R5 (end group) being a substituent which is dependent on the termination reaction, for example hydroxyl, NH2, NHR or NR2, with the R radicals being able to correspond to the end groups R3 and R4,

and with the average degree of polymerization P=(m+n) being in the range from 45 to 5250, preferably in the range from 250 to 2250, particularly preferably in the range from 500 to 2050, and n=a×P with 0.0001<a<0.1, preferably 0.01<a<0.05, and particularly preferably a=0.03.

In this connection, the units m and n are not block structures but, instead, distributed randomly in the polymer.

Another polyethyleneimine which can preferably be used for producing the medicaments possesses the following general formula:

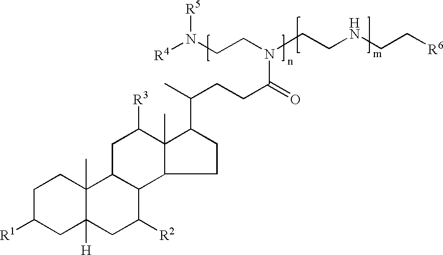

in which, in each individual [CH2—CH2—N] unit,

R1, R2 and R3 denote hydrogen or hydroxyl,

and in which

R4 and R5 (end groups) denote, independently of each other, hydrogen or steroid parent substances, such as bile acids, or possess a structure which is dependent on the initiator,

with R6 (end group) being a substituent which is dependent on the termination reaction, for example hydroxyl, NH2, NHR or NR2, with the R radicals being able to correspond to the end groups R4 and R5,

and with the average degree of polymerization P=(m+n) being in the range from 45 to 5250, preferably in the range from 250 to 2250, particularly preferably in the range from 500 to 2050, and n=a×P with 0.0001<a<0.1, preferably 0.01<a<0.05, and, particularly preferably, a=0.03.

In this connection, all stereoisomers with regard to the steroid backbone chain are included. In particular, the substituents R1, R2 and, R3 can be arranged both in the a configuration and in the β configuration. In the same way, the substituent in the 5 position can be present in, the α configuration and in the β configuration (nomenclature in accordance with Römpp-Chemielexikon [Römpp chemical encyclopedia], 9th edition, Georg Thieme Verlag, 1992).

In this connection, the units m and n are not block structures but, instead, distributed randomly in the polymer.

Another polyethyleneimine which can preferably be used for producing the medicaments possesses the following general formula:

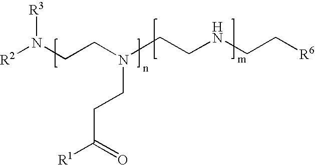

in which, in each individual [CH2—CH2—N] unit,

R1 denotes OR4 or NR4R5,

with

R4 and R5 denoting, independently of each other, hydrogen or alkyl having from 1 to 24 carbon atoms, preferably alkyl having from 13 to 24 carbon atoms, particularly preferably alkyl having 18 carbon atoms,

and in which

R2 and R3 (end groups) correspond, independently of each other, to the substituents of the nitrogen atoms of the main polymer chain or possess a structure which is dependent on the initiator,

with R6 (end group) being a substituent which is dependent on the termination reaction, for example hydroxyl, NH2, NHR or NR2, with the R radicals being able to correspond to the end groups R2 and R3.

and with the average degree of polymerization P=(m+n) being in the range from 45 to 5250, preferably in the range from 250 to 2250, particularly preferably in the range from 500 to 2050, and n=a×P with 0.0001<a<0.1, preferably 0.01<a<0.05, and, particularly preferably, a=0.03.

In this connection, the units m and n are not block structures but, instead, randomly distributed in the polymer.

Another polyethyleneimine which can preferably be used for producing medicaments possesses the following general formula:

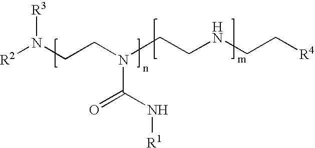

in which, in each individual [CH₂—CH₂—N] unit,

R¹ denotes alkyl having from 1 to 24 carbon atoms, preferably alkyl having from 13 to 24 carbon atoms, particularly preferably alkyl having 18 carbon atoms,

and in which

R² and R³ (end groups) correspond, independently of each other, to the substituents of the nitrogen atoms of the main polymer chain or possess a structure which is dependent on the initiator,

with R⁴ (end group) being a substituent which is dependent on the termination reaction, for example hydroxyl, NH₂, NHR or NR₂, with the R radicals being able to correspond to the end groups R² and R³,

and with the average degree of polymerization P=(m+n) being in the range from 45 to 5250, preferably in the range from 250 to 2250, particularly preferably in the range from 500 to 2050, and n=a×P with 0.0001<a<0.1, preferably 0.01<a<0.05, and, particularly preferably, a=0.03.

In this connection, the units m and n are not block structures but, instead, randomly distributed in the polymer.

Another polyethyleneimine which can preferably be used for producing the medicaments possesses the following general formula:

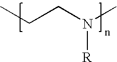

in which, in each individual. [CH₂—CH₂—N] unit, the radical R can be either hydrogen or a radical of the formula

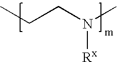

and in which R^{x} can be either hydrogen or also, once again, a radical of the type R,

and in which each individual [CH₂—CH₂—N] unit, and the end groups, can carry the abovementioned substituents,

and with the average degree of polymerization P=(m+n) being in the range from 45 to 5250, preferably in the range from 250 to 2250, particularly preferably in the range from 500 to 2050, and n=a×P with 0.0001<a<0.1, preferably 0.01<a<0.05, and, particularly preferably a=0.03. These polyethyleneimines have a branched or crosslinked structure.

The polymer preferably has an average molecular weight of less than 220000 g/mol, particularly preferably a molecular weight of between 2000 and 100000 g/mol, very particularly preferably a molecular weight of between 20000 and 100000 g/mol.

The hydrophobic groups are introduced in polymer-analogous reactions, for example by alkylating with halogenoalkanes, acylating with carbonyl chlorides, acylating with reactive esters, Michael addition to α,β-unsaturated carbonyl compounds (carboxylic acids, carboxamides, carboxylic esters) or by addition to isocyanates. These are reaction types which are known from the literature (March, 1992).

The linear polyethyleneimines are prepared, for example, by the cationic ring opening polymerization of 2-ethyloxazoline using cationic initiators, preferably in accordance with a protocol by B. L. Rivas and S. I. Ananias (1992). The poly(ethyloxazolines) which are obtained in this way are converted quantitatively into the linear polyethyleneimines by being treated with a mixture composed of concentrated hydrochloric acid and water, preferably a 1:1 mixture of concentrated hydrochloric acid and water, with propanoic acid being eliminated. The reaction temperature is preferably between 80 and 100° C., particularly preferably 100° C. The reaction time is preferably between 12 and 30 hours, particularly preferably 24 hours. The product is preferably purified by being crystallized several times from ethanol.

The process which has been described can be used to prepare the linear polyethyleneimines in the desired molecular weight range of from 2000 to 220000 g/mol.

The alkyl groups, such as C18-alkyl groups, are introduced, for example; by reacting a 5% solution of the appropriate linear polyethyleneimine with octadecyl chloride in absolute ethanol at a reaction temperature of from 40 to 75° C., preferably 60° C. The quantity of the alkyl chloride which is metered in is geared precisely to the desired degree of substitution (from 0.1 to 10%). The reaction time is preferably between 10 and 24 hours, particularly preferably 17 hours.

Acyl groups, such as C18-acyl groups, are introduced, for example, by reacting a 5% solution of the appropriate linear polyethyleneimine with octadecanoyl chloride in absolute ethanol at a reaction temperature of from 40 to 60° C., preferably 50° C. The quantity of the acid chloride which is metered in is geared precisely to the desired degree of substitution (from 0.1 to 10%). The reaction time is preferably between 10 and 24 hours, particularly preferably 20 hours.

A reactive ester method can also be used to introduce acyl groups, with a carboxylic acid derivative being activated with N-hydroxysuccinimide. Preference is given to using this method when functionalizing the polyethyleneimine with bile acid. For this, the bile acid derivative chenodeoxycholic acid (3α,7α-dihydroxy-5β-cholanic acid), abbreviated as a substituent to CDC in that which follows, is, for example, reacted with N-hydroxysuccinimide in dimethoxyethane as the solvent and in the presence of dicyclohexylcarbodiimide. The reaction is carried out at room temperature and the reaction time is 16 hours. The reactive ester which has been prepared in this way is reacted with a 5% solution of the appropriate linear polyethyleneimine in absolute ethanol. The quantity of the reactive ester which is metered in is geared precisely to the desired degree of substitution (from 0.1 to 10%). The reaction temperature is between 20 and 60° C., preferably 50° C. The reaction time is preferably between 10 and 24 hours, particularly preferably 20 hours.

The use of the reactive ester method to introduce, for example, chenodeoxycholic acid into oligoamines, such as spermine or pentaethylenehexamine, is described in the literature (Walker et. al., 1998). The bile acid-substituted polymers according to the invention possess hydrophobic substituents, with it being possible to use the number of the hydroxyl groups to control the degree of the hydrophobicity, in an analogous manner to that in the cationic facial amphiphiles described by S. Walker et al.

Highly purified samples are prepared by dissolving the polyamines, and in particular the hydrophobic polyethyleneimines, in water at pH 7 and at a concentration of from 0.1 to 1 mg/ml, preferably 0.5 mg/ml, and purifying them by column chromatography through Sephadex and subsequent freeze-drying. The polymers are then once again dissolved in water, or preferably physiological sodium chloride solution, while being briefly sonicated, and adjusted to pH 7. The concentration of the polyamine or polyethyleneimine stock solutions is preferably between 0.1 and 1 mg/ml, particularly preferably 0.5 mg/ml. The stock solutions are stable during storage at room temperature; they are preferably stored at 4° C.

It is possible to use standard methods, such as 1H NMR spectroscopy, FT-IR spectroscopy and zeta potential measurements for characterizing the cationic polymers.

The polyamines which can be used for producing the medicaments can also be coupled to cell-specific ligands. These cell-specific ligands can, for example, be constituted such that they bind to the outer membrane of a target cell, preferably of an animal or human target cell. The target cell can, for example, be an endothelial cell, a muscle cell, a macrophage, a lymphocyte, a glial cell, a hemopetic cell, a tumor cell, for example a leukemia cell, a virus-infected cell, a bronchial epithelial cell or a liver cell, for example a sinusoidal cell of the liver. A ligand which specifically binds to endothelial cells can be selected, for example, from the group consisting of monoclonal antibodies or their fragments which are specific for endothelial cells, glycoproteins carrying manhose terminally, glycolipids or polysaccharides, cytokine, growth factors or adhesion molecules, or, in a particularly preferred embodiment, of glycoproteins from the envelopes of viruses which have a tropism for endothelial cells. A ligand which binds specifically to smooth muscle cells can be selected, for example, from the group which comprises monoclonal antibodies or their fragments which are specific for actin, cell membrane receptors and growth factors or, in a particularly preferred embodiment, from glycoproteins derived from the envelopes of viruses which have a tropism for smooth muscle cells. A ligand which binds specifically to macrophages and/or lymphocytes can be selected, for example, from the group comprising monoclonal antibodies which are specific for membrane antigens on macrophages and/or lymphocytes, intact immunoglobulins or Fc fragments of polyclonal or monoclonal antibodies which are specific for membrane antigens or macrophages and/or lymphocytes, cytokines, growth factors, peptides which carry mannose terminally, proteins, lipids or polysaccharides or, in a particularly preferred embodiment, from glycoproteins which are derived from the envelopes of viruses, in particular the HEF protein of the influenza C virus which has a mutation in nucleotide position 872 or influenza C virus HEF cleavage products which contain the catalytic triad serine 71, histidine 368 or 369 and aspartic acid 261. A ligand which binds specifically to glial cells can be selected, for example, from the group which comprises antibodies and antibody fragments which bind specifically to glial cell membrane structures, adhesion molecules, peptides which carry mannose terminally, proteins, lipids or polysaccharides, growth factors or, in a particularly preferred embodiment, from glycoproteins which are derived from the envelopes of viruses which have a tropism for bile cells. A ligand which binds specifically to hematopetic cells can be selected, for example, from the group which comprises antibodies or antibody fragments which are specific for a stem cell factor receptor, IL-1 (in particular receptor type I or II), IL-3 (in particular receptor type α or β), IL-6 or GM-CSF, and also intact immunoglobulins or Fc fragments which exhibit this specificity and growth factors such as SCF, IL-1, IL-3, IL-6 or GM-CSF, and their fragments, which bind to the a pertinent receptors. A ligand which binds specifically to leukemia cells can be selected, for example, from the group which comprises antibodies, antibody fragments, immunoglobulins or Fc fragments which bind specifically to membrane structures on leukemia cells, such as CD13, CD14, CD15, CD33, CAMAL, sialosyl-Le, CD5, CD1e, CD23, M38, IL-2 receptors, T cell receptors, CALLA or CD19, and also growth factors, or fragments which are derived therefrom, or retinoids. A ligand which binds specifically to virus-infected cells can be selected, for example, from the group which comprises antibodies, antibody fragments, intact immunoglobulins or Fc fragments which are specific for a virus antigen which, following infection with the virus, is expressed on the cell membrane of the infected cell. A ligand which can bind specifically to bronchial epithelial cells, sinusoidal cells of the liver or liver cells, can be selected, for example, from the group which comprises transferin, asialoglycoproteins, such as asialoorosomucoid, neoglycoprotein or galactose, insulin, peptides which carry mannose terminally, proteins, lipids or polysaccharides, intact immunoglobulins or Fc fragments which bind specifically to the target cells and, in a particularly preferred embodiment, from glycoproteins which are derived from the envelopes of viruses which bind specifically to the target cells. Other detailed examples of ligands are disclosed, for example, in EP-A 0 790 312 and EP-A 0 846 772.

In general, the medicaments according to the invention comprise between 0.5 and 500 mg of polyamine per dose as the active substance, preferably between 20 and 100 mg per dose.

In addition to the polyamine as active substance, the medicaments according to the invention can also comprise other pharmaceutical active compounds, such as Parapox ovis (for example in the form of BAYPAMUN®, fragments of Parapox ovis, CpG-containing oligonucleotides, antibiotics and cytostatic agents.

The polyamines which can be used for producing the medicaments according to the invention, or the medicaments according to the invention themselves, are preferably present, after the polyamines have been purified and lyophilized, in solid form and can then be dissolved in a suitable aqueous medium, preferably physiological sodium chloride solution, immediately prior to administration, or be administered directly in a suitable formulation with which additives have been admixed, where appropriate after dispersing in aqueous media, preferably in physiological sodium chloride solution.

Other formulation aids which are suitable are biocompatible and biodegradable polymers such as polylactide, polylactidecoglycolide, polyacrylates, polyorthoesters, polyanhydrides, polyamides, polyamino acids, cellulose derivatives, starch derivatives or chitosan derivatives.

Depending on the clinical problem, the polyamine-based medicament is either administered systemically (e.g. orally, intramuscularly, subcutaneously. intraperitoneally or intravenously) or locally (for example into the organ concerned).

Several administrations, or long-term treatment in accordance with timetables which correspond to the requirements of the clinical problem, may be necessary in this connection.

Particularly on account of their immunostimulatory, antiinflammatory and antiapoptotic effect (cytokine induction and overexpression of antiinflammatory and antiapoptotic factors), polyamines can be used for treating the following diseases/lesions or for the prophylaxis/metaphylaxis of the following diseases:

viral infections

On the basis of the known connections between the influence of a T1 immune response on latent, chronic, persistent viral infections (Lucin et al., 1994; Smith et al., 1994) and an effect of the polyamine which is comparable to that of the immunostimulator Parapox ovis, it is possible, and of therapeutic value, to use polyamines, as a monotherapy or in combination with biologically active, for example antiviral, low molecular weight compounds, in humans and animals for the antiviral therapy of infections with hepatitis B virus, hepatitis C virus or any of the other pathogens from the group of the hepatitis-causing viruses, and also other viral infections of the internal organs, and also infections, including those which are accompanied by other diseases, with the different types of herpes simplex virus (HSV), the different types of human papilloma virus (HPV), human immunodeficiency virus (HIV) and human cytomegalovirus (HCMV), and also the corresponding viral diseases in animals.

Acute or chronic viral infections of the respiratory tract and the internal organs

Infections due to stress or following an operation or dental treatment

Bacterial infections, in particular infections with intracellular bacteria

Cancer, tumors

Organ fibroses, in particular liver fibrosis or liver cirrhosis following viral hepatitis or ethanol-induced liver diseases and also cystic fibrosis

Diseases which are accompanied by an increased deposition of collagen, in connection with which both the internal organs, for example the liver, and the skin, and its appendages, can be affected

Inflammatory, degenerative and proliferative diseases of the internal organs, the skin, the blood or the central nervous system and its appendages, including the eye

The group of allergic diseases, in particular for preventing the onset of systemic allergies or for use in connection with topical allergies or asthma.

The polyamines can also be used as adjuvants.

### EXAMPLES

### Example 1

### Synthesizing the Linear Polyethyleneimines (LPEI)

Linear polyethyleneimines were synthesized by cationic ring-opening polymerization of 2-ethyloxazoline to give poly(ethyloxazoline) (in analogy with Rivas & Ananias, 1992) and subsequent acid hydrolysis, with the elimination of propanoic acid. Certain precursor polymers (poly(ethyloxazolines)) can also be obtained commercially (Sigma-Aldrich Chemie GmbH, Germany). The precursor polymers were characterized by gel permeation chromatography, 1H NMR and FT-IR.

Quantitative hydrolysis was achieved by reacting 24.7 g, for example, of poly(ethyloxazoline) (Mw 200000 g/mol) at 100° C. in a mixture consisting of 40 ml of water and 40 ml of concentrated hydrochloric acid. After 24 hours, the voluminous precipitate which had formed was dissolved by, adding 250 ml of water. After it had been cooled down to 20° C., the product was adjusted to pH 11, by adding 20% NaOH, and precipitated. After the precipitate had been filtered off with suction and washed (washing water, pH 7), it was dried over phosphorus pentoxide under high vacuum. The crude product was then recrystallized from ethanol (yield 9.5 g/88%). Highly pure batches (milligram quantities) were obtained by subjecting saturated aqueous solutions (pH 7) of the polyethyleneimine to column chromatography through Sephadex G25 (Pharmacia disposable PD-10 desalting column), using Millipore water as eluent, and then freeze-drying.

The linear polyethyleneimines were characterized by 1H NMR and FT-IR, thereby making it possible to confirm that the hydrolysis was quantitative.

### Example 2

### Synthesizing the Hydrophobically Functionalized Linear Polyethyleneimines (H-LPEIs), Taking as an Example the Introduction of 3 mol % of C18-alkyl Groups into LPEI Having a Mw of 87000 g/Mol

For this, 0.5 g of LPEI were dissolved, at 60° C. and under argon, in 10 ml of ethanol and, after 0.11 g (0.13 ml) of octadecyl chloride had been slowly added, the mixture was stirred for 17 hours. The reaction product was precipitated at 20° C. by adding 20 ml of water, then filtered off, washed with water (washing water, pH 7) and dried over phosphorus pentoxide under high vacuum (yield 0.48 g/96%). Highly pure batches (milligram quantities) were obtained by subjecting saturated aqueous solutions (pH 7) of the polyethyleneimine to column chromatography through Sephadex G25 (Pharmacia disposable PD-10 desalting column) using Millipore water as eluent and then freeze-drying.

The alkylated linear polyethyleneimines were characterized by 1H NMR and FT-IR, thereby making it possible to confirm that the desired degree of alkylation had been obtained.

### Example 3

### Synthesizing the Hydrophobically Functionalized Linear Polyethyleneimines (H-LPEIs), Taking as an Example the Introduction of 3 mol % of C18-acyl Groups into LPEI Having a Mw of 87000 g/Mol

For this, 0.5 g of LPEI was dissolved, at 50° C. and under argon, in 10 ml of ethanol and, after 0.11 g (0.12 ml) of octadecanoyl chloride had been slowly added, the mixture was stirred for 20 hours. After filtration, the reaction mixture was quantitatively concentrated in vacuo. The residue was dissolved in 4 ml of ethanol in the hot and the product was precipitated, at 20° C., by adding 8 ml of water. After it had been filtered and washed with water (washing water, pH 7), the precipitate was dried over phosphorus pentoxide under high vacuum (yield 0.38 g/76%). Highly pure batches (milligram quantities) were obtained by subjecting saturated aqueous solutions (pH 7) of the polyethyleneimine to column chromatography through Sephadex G25 (Pharmacia disposable PD-10 desalting column) using Millipore water as eluent, and then freeze-drying.

The acylated linear polyethyleneimines were characterized by 1H NMR and FT-IR, thereby making it possible to confirm that the desired degree of acylation had been obtained.

### Example 4

### Synthesizing the hydrophobically Functionalized Linear Polyethyleneimines (H-LPEIs), taking as an Example the Introduction of 3 mol % of Chenodeoxycholic Acid (CDC) Groups (3α,7α-dihydroxy-5β-cholanic acid) into LPEI Having a Mw of 87000 g/Mol

For this, N-hydroxysuccinimide was used to convert chenodeoxycholic acid (Sigma-Aldrich Chemie GmbH) into a reactive ester compound. 1 g of chenodeoxycholic acid and 0.32 g of N-hydroxysuccinimide were dissolved in 5 ml of dimethoxyethane and reacted, at 0-5° C., with 0.63 g of dicyclohexylcarbodiimide. The reaction mixture was stirred for 16 hours, after which the precipitate was filtered off and the filtrate was concentrated in vacuo. The reactive ester was dried under high vacuum (stable foam) and characterized by 1H NMR. Without any further purification, 179 mg of the chenodeoxycholic acid reactive ester were added, at room temperature and under argon, to a solution of 0.5 g of LPEI in 10 ml of ethanol. The reaction mixture was then stirred at 50° C. for 20 hours. After the mixture had been cooled down to room temperature, the product was precipitated by adding 25 ml of water. The residue was filtered off, washed with water (washing water, pH 7) and dried over phosphorus pentoxide under high vacuum. (Yield 0.41 g/82%). Highly pure batches (milligram quantities) were obtained by subjecting saturated aqueous solutions (pH 7) of the polyethyleneimine to column chromatography through Sephadex G25 (Pharmacia disposable PD-10 desalting column) using Millipore water as eluent, and then freeze-drying.

The linear polyethyleneimines which had been acyl-functionalized using the reactive ester method were characterized by 1H NMR and FT-IR, thereby making it possible to confirm that the desired degree of acylation had been obtained.

### Example 5

### Zeta Potential Measurements

Zeta potential measurements were carried out for determining the charge or the degree of protonation of the linear polyethyleneimines, and of the hydrophobically functionalized polyethyleneimines, in aqueous solution and at a physiological pH. Independently of the average molecular weight, and independently of the polymer type, the average degree of protonation was found to be 50% at pH 7, i.e., in aqueous solution at pH 7, approx. 50% of the nitrogen atoms are protonated.

### Example 6

Stock solutions of all the polyethyleneimines (LPEIs, H-LPEIs) were prepared in physiological sodium chloride solution at pH 7, with the concentration of the polyethyleneimine in each case being 0.5 mg/ml. For this, 25 mg of the LPEI or the H-LPEI were dissolved, while heating and briefly sonicating, in 30 ml of water or physiological sodium chloride solution; the resulting solution was then adjusted to pH 7 with 0.1 N HCl and made up to a final volume of 50 ml. The stock solutions were sterilized by filtration (0.2 μm) and can be stored at 20° C. for a long period.

In order to substantiate the suitability of polyamines, in particular polyethyleneimines, for use as immunotherapeutic agents, the interferon γ-stimulating effect of the polyamines was demonstrated in vivo, with the in vivo efficacy of the polyamines then being demonstrated.

### 1. Inducing IFN-γ in a Mouse Spleen Cell Assay

### a) Animal Management

The NMRI mice (outbred strain, female, weight 18-20 g) were obtained from Charles River (Sulzfeld, Germany) 8 days before the experiments began. The animals had free access to feed and water and were maintained in an artificial day/night rhythm (illumination from 07:00 to 19:00 hours, darkness from 19:00 to 07:00 hours).

### b) Preparing Mouse Spleen Cells

The animals were sacrificed by cervical dislocation and the spleens were then removed. The spleens were freed from adhering connective tissue and worked up in accordance with the following protocol:

The spleen is laid (Petri dish) on a metal sieve (mesh width approx. 70 μm) and minced using a pair of scissors. 5 ml of PBS are then added and the tissue pieces are pressed through the sieve using a glass pestle. The sieve is subsequently rinsed several times with PBS, after which the cells, in a total of 50 ml PBS, are transferred to a Falcon tube and then centrifuged at 300×g for 10 minutes. The supernatant is discarded and the cells are resuspended in 20 ml of PBS and then centrifuged once again at 300×g for 10 minutes. After the cells have been resuspended in 5-10 ml of medium, the cell count is determined and adjusted with medium to 2.5×10⁶ cells/ml.

### c) Stimulating Mouse Spleen Cells

The stimulations took place, at 37° C. and 5% CO₂ for 72 hours, in a volume of 1 ml in 24-well plates. 2×10⁶ spleen cells were stimulated in a total volume of 1 ml (0.8 ml of medium: RPMI, 10% FCS, 1% penicillin/streptomycin). Depending on the number of stimulants, the following were mixed together for each assay:

800 μl of medium containing 2.5×10⁶ cells/ml

100 μl of stimulant (polyethyleneimine, 0.5 mg/ml)

100 μl of PBS

All the spleen cell stimulations were carried out in duplicate.

After the stimulation had ended, the supernatants were transferred to 1.5 ml reaction tubes and the remaining cells were separated off by centrifuging at 300×g for 10 minutes. The cell-free supernatants were removed and stored at −20° C. until the IFN-γ was measured by ELISA.

### d) Measuring the IFN-γ Concentrations

The mouse IFN-γ OptEIA®-ELISA set (Pharmingen, Heidelberg, Germany) was used, in accordance with the manufacturer's instructions, for determining the concentrations of IFN-γ in the stimulation supernatants.

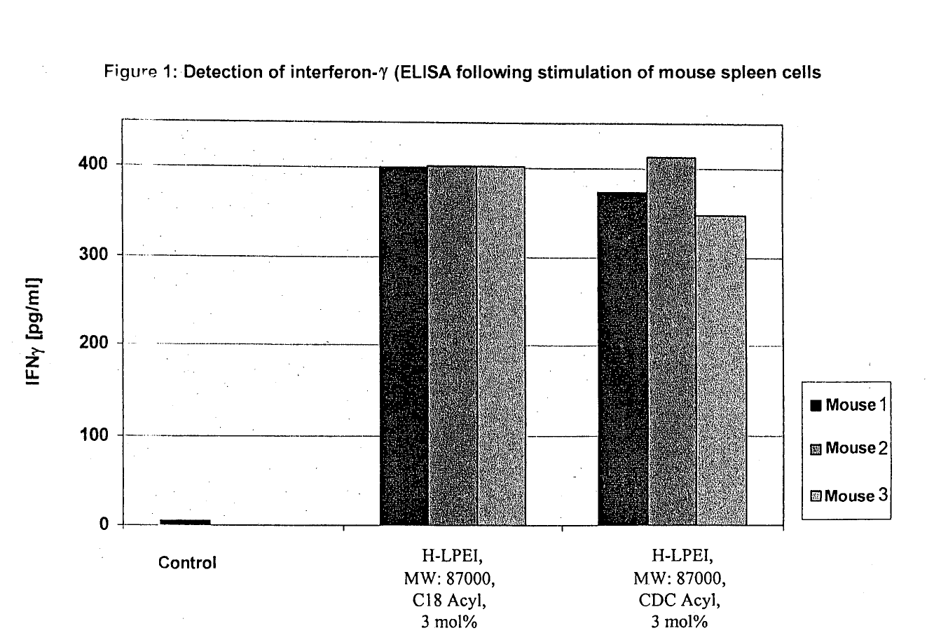

Result as depicted in FIG. 1:

The tested polyethyleneimines show a significant induction of IFN-γ in the mouse spleen cell assay.

### 2. Inducing IFN-γ In Vivo

### a) Mouse Management

NMRI mice (outbred strain HdsWin:NMRI, female, weight 18-20 g, obtained from Harlan/Winkelmann, Borchen, Germany) were kept, in autoclavable, wood shaving-lined polycarbonate boxes, in an S2 isolation barn at 20-22° C. (atmospheric humidity 50-60%) and in an artificial day/night rhythm (illumination from 06:30 to 18:30 hours, darkness from 18:30 to 06:30 hours). They had free access to feed and water.

### b) Implementing the Experiment

The animals were randomized and divided up into two groups of in each case 6 animals. Following their arrival, the mice were initially kept for 3 days in hutches without any further treatment.

The substances to be analyzed were administered intraperitoneally in a volume of 0.2 ml. The following treatment scheme was employed:

| | |
|---|---|
| Group 1: | placebo: PBS |
| Group 2: | polyethyleneimine, H-LPEI, M_{w}: 87000, C18 acyl, 3 mol %, |
| | in accordance with Example 3 (0.1 mg per mouse). |

9 hours after the treatment, the mice were sacrificed and the peritoneal cells were obtained by rinsing the abdomen with 5 ml of ice-cold PBS.

The cells were concentrated by means of a centrifugation step (30 seconds at 16,000×g, room temperature), after which the supernatant was poured off and the total RNA in the cells was extracted using the NucleoSpin RNA II kit (Machery-Nagel, Düren, Germany).

For this, the cell pellet was resuspended in 400 μl of RA1 buffer (from the NucleoSpin RNA II kit) and frozen at −80° C. After it had been thawed at 37° C., the mixture was loaded, for the purpose of reducing its viscosity, onto a NucleoSpin filter and centrifuged for 1 minute (16,000×g, room temperature). 300 μl of ethanol were added to the filtrate and the mixture was loaded onto a NucleoSpin RNA column. After centrifuging (30 seconds at 8,000×g), removing the filtrate and then subjecting the column to centrifugal drying (1 minute, 16,000×g), the DNA was cleaved with DNase I. For this, 90 μl of DNase reaction buffer were mixed with 10 μl of DNase I (both obtained from the NucleoSpin RNA II kit) and 95 μl of this. solution were applied to the dry filter. After incubating for 15 minutes (room temperature), the filter was firstly washed with 500 μl of RA2, then with. 600 μl of RA3 and subsequently with 250 μl of RA3 (obtained from the NucleoSpin RNA II kit). To do this, the washing buffers were applied and the column was in each case centrifuged for 30 seconds at 8,000×g and then, after the last washing step, centrifuged for 2 minutes at 16,000×g. After that, the RNA was eluted in 60 μl of RNase-free, distilled water (1 minute at 16,000×g).

The quality of the RNA was checked by photometric measurement.

The cDNA was synthesized by reverse-transcribing the RNA using random hexamers as primers for the polymerase reaction. Use was made of the TaqMan reverse transcription reagent (Applied Biosystems, Weiterstadt, Germany) in this connection. The synthesis was carried out in 100 μl.

The composition of the synthesis mixture was as follows:

1300-1500 μg of RNA

10 μl of RT buffer (10×)

22 μl of MgCl₂ (25 mM)

5 μl of random hexamers

2.5 μl of MultiScribe RT

2 μl of RNase inhibitor

20 μl of dNTP

RNase-free water (to make up to a total volume of 100 μl)

The mixture was incubated in a GeneAmp 2400 Thermocycler (Applied Biosystems, Weiterstadt, Germany), initially for 10 minutes at 25° C. and then for 30 minutes at 48° C.; it was then cooled down to +4° C. The cDNA which was synthesized in this way was stored at −20° C.

Quantitative PCR was carried out using a PRISM® 5700 ABI (Applied Biosystems, Weiterstand, Germany). The PDAR (predeveloped TaqMan® assay reagent) kit for murine. IFN-γ (Applied Biosystems, Weiterstadt, Germany) was used for this purpose.

The PDAR kit was used to normalize the quantities of the cDNAs with the aid of a housekeeping gene (18S RNA), “endogenous control ribosomal RNA control (18S RNA)”.

A calibrator cDNA was used for standardizing and calculating the induction. This cDNA consisted of a mixture of the cDNA from 11 different mice, which were treated in accordance with group 1.

The amplifications were in each case carried out in a volume of 50 μl and their compositions were as follows:

Endogenous 18S RNA control:

1 ng of cDNA

25 μl of 2×TaqMan Universal PCR Master Mix

2.5 μl of 18S RNA

RNase-free water (to make up to a total volume of 100 μl)

Detection of IFN-γ:

10 ng of cDNA

25 μl of 2×Taqman Universal PCR Master Mix

2.5 μl of IFN-γ primers

RNase-free water (to make up to a total volume of 100 μl)

After incubating at 50° C. for 2 minutes, and the subsequent initial denaturation (95° C., 10 minutes), there then followed 45 cycles of denaturation (94° C., 15 seconds). and annealing/extension (60° C., 1 minute). The GeneAmp® 5700 Sequence Detection System Software (v. 1.3) was used for analyzing the products.

The following results were obtained:

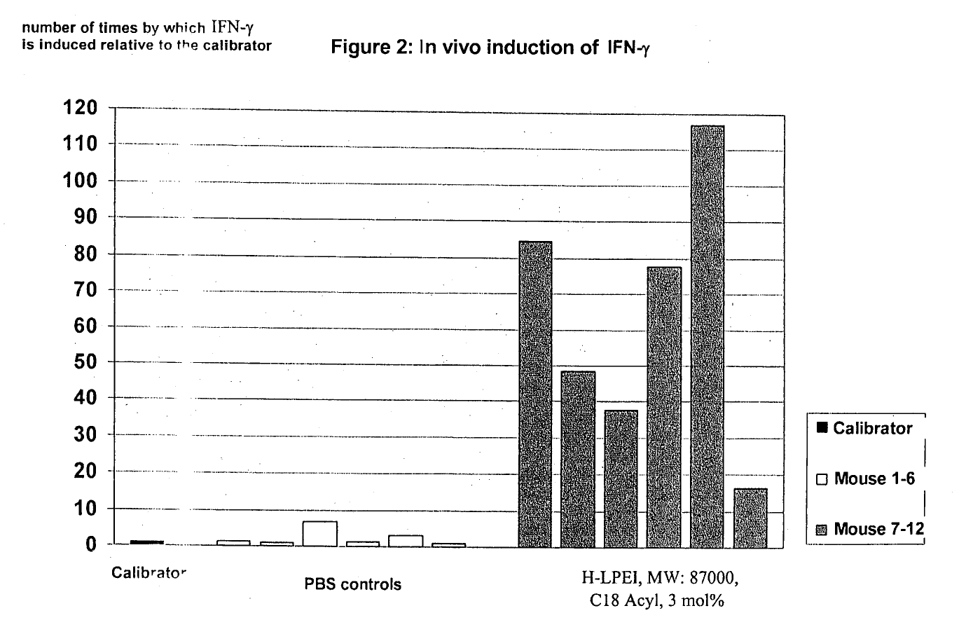

Expression of interferon-γ is induced in vivo 9 hours after treating with polyethyleneimine H-LPEI, M_{w}: 87000, C18 acyl, 3 mol % (cf. FIG. 2). Depending on the animal, this induction is 16-120-fold higher than the calibrator. On the other hand, the untreated animals, or the animals treated with PBS, exhibit an expression of interferon-γ which varies from 0.9 to 7 times that of the calibrator.

### 3. Detecting the Immunostimulatory Effect in the Aujeszky Mouse Model

The Aujeszky mouse model is an in vivo stress model for detecting the effect of different immunostimulators (e.g. BAYPAMUN® and CpG-oligonucleotides).

### a) Mouse Management

NMRI mice (outbred strain HdsWin:NMRI, female, weight 18-20 g, obtained from Harlan/Winkelmann, Borchen, Germany) were kept, in autoclavable, wood shaving-lined polycarbonate boxes, in an S2 isolation barn at 20-22° C. (atmospheric humidity 50-60%) and in an artificial day/night rhythm (illumination from 06:30 to 18:30 hours, darkness from 18:30 to 06:30 hours). They had free access to feed and water.

### b) Stress Model

For the investigations, mouse groups were formed containing 10 mice per group. The animals in any one group were in each case given the same test substance.

Following their arrival, the mice were kept in hutches for 2-3 days. Subsequently, the polyethyleneimines (starting concentration 0.5 mg/ml) were diluted 1:10 and 1:100 with physiological NaCl solution (pH 7.6). These solutions were administered intraperitoneally at the rate of 0.2 ml per mouse.

24 hours after the treatment, the mice were 'stressed by the intraperitoneal administration of pseudorabies virus, strain Hannover H2. For this, the virus was diluted in PBS to give a stress titer of 10^{3.8}-10^{4.1} TCID₅₀/ml, and 0.2 ml of this suspension was then administered.

As a negative control, a group of mice was treated with physiological NaCl solution and then stressed.

The mice in this group died 3-8 days after having been stressed. A large proportion of the polyethyleneimine-treated mice survived the infection with pseudorabies virus. The experiment was terminated 10 days after stressing.

The strength of the induced immunostimulation was determined by comparing the mice which had died in the NaCl control group and the test groups and was quantified by means of the efficacy index. This specifies the percentage number of mice which are protected from the lethal effect of the Aujescky virus as a result of having been immunostimulated with the substance being tested. It is calculated using the formula

EI=(b−a)/b×100.

In this formula, b specifies the percentage of dead mice in the control group while a specifies the percentage of dead mice in the test group.

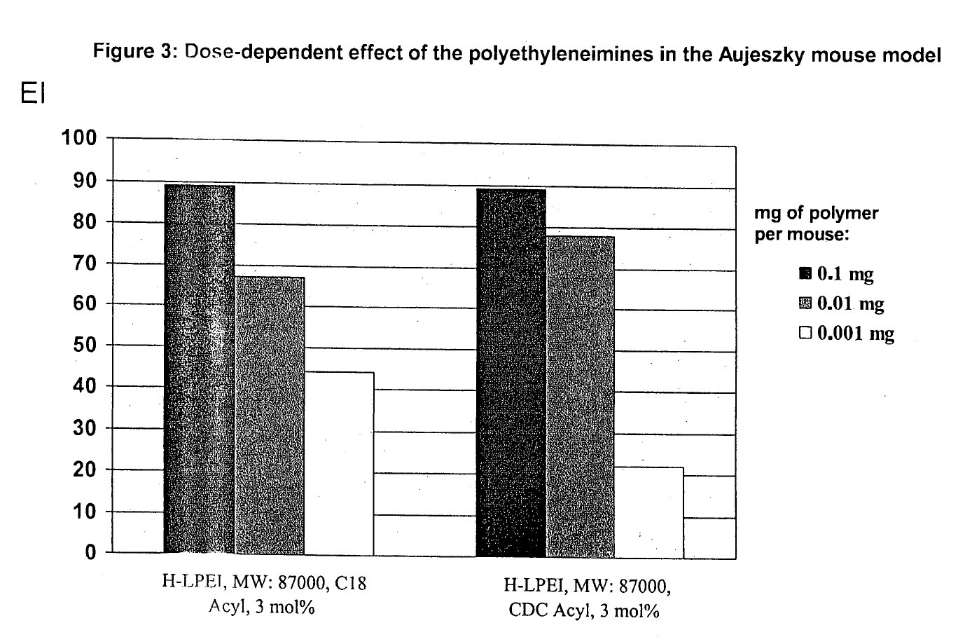

Results (cf. FIG. 3):

| | Substance, concentration, | Σ of dead | Efficacy index |
|---|---|---|---|
| | Quantity administered per mouse | animals | EI |
| 1. | NaCl | 9 | — |
| 2. | H—LPEI, M_{w}: 87000, C18 acyl, 3 mol %, 0.5 mg/ml, | 1 | 89 |
| | (0.1 mg per mouse) | | |
| 3. | H—LPEI, M_{w}: 87000, C18 acyl, 3 mol %, 0.05 mg/ml, | 3 | 67 |
| | (0.01 mg per mouse) | | |
| 4. | H—LPEI, M_{w}: 87000, C18 acyl, 3 mol %, 0.005 mg/ml, | 5 | 44 |
| | (0.001 mg per mouse) | | |
| 5. | H—LPEI, M_{w}: 87000, CDC acyl, 3 mol %, 0.5 mg/ml, | 1 | 89 |
| | (0.1 mg per mouse) | | |
| 6. | H—LPEI, M_{w}: 87000, CDC acyl, 3 mol %, 0.05 mg/ml, | 2 | 78 |
| | (0.01 mg per mouse) | | |
| 7. | H—LPEI, M_{w}: 87000, CDC acyl, 3 mol %, 0.005 mg/ml, | 7 | 22 |
| | (0.001 mg per mouse) | | |

Testing different concentrations of polyethyleneimines in the Aujeszky mouse model surprisingly demonstrates the following:

A significant immunostimulation, with efficacy indices of ≧60%, was in each case demonstrated after treating the mice with 0.1 or 0.01 mg of H-LPEI, M_{w}: 87000, C18 acyl, 3 mol %, or H-LPEI, M_{w}: 87000, CDC acyl, 3 mol %.

A dose/effect relationship was in each case demonstrated when using different concentrations of the two polyethyleneimines H-LPEI, M_{w}: 87000, C18 acyl, 3 mol % and H-LPEI, M_{w}: 87000, CDC acyl, 3 mol %.

### 4. Using the PIQOR™ cDNA Array System to Analyze the Effect of the Polyethyleneimines H-LPEI M_{w}: 87000. C18 Acyl, 3 mol %, and H-LPEI, M_{w}: 87000. CDC Acyl 3 mol % on Gene Expression in Murine Peritoneal Cells (In Vivo)

### a) Animal Management

The NMRI mice (outbred strain, female, weight 18-20 g) were obtained from Charles River (Sulzfeld, Germany) 8 days before beginning the experiments. The animals had free access to feed and water and were kept in an artificial day/night rhythm (illumination from 07:00 to 19:00 hours, darkness from 19:00 to 07:00 hours).

### b) Experimental Procedure

The animals were randomized and divided into three groups of in each case 4 animals. The substances to be analyzed were administered intraperitoneally in a volume of 0.2 ml. The following treatment scheme was used:

| | |
|---|---|
| Group 1: | Placebo: physiological NaCl solution. |
| Group 2: | polyethyleneimine, H—LPEI, M_{w}: 87000, C18 acyl, 3 mol % |
| | (0.1 mg per mouse). |
| Group 3: | polyethyleneimine, H—LPEI, M_{w}: 87000, CDC acyl, 3 mol |
| | % (0.1 mg per mouse). |

The mice were sacrificed 6 hours after the treatment and the peritoneal cells were isolated by lavaging the abdomen with 10 ml of medium (DMEM, 5% FCS).

The cells were subsequently concentrated by centrifugation (10 minutes at 300×g, room temperature) and then either used immediately for the RNA extraction or first of all subjected to lysis of the erythrocytes.

### Erythrocyte Lysis

For the erythrocyte lysis, the pelleted peritoneal cells were resuspended in 1 ml of PBS, after which 10 ml of lysis buffer (10 ml of 0.17 M Tris, pH 7.2+90 ml of 0.16 M NH₄Cl, pH 7.2) were added and the mixture was incubated at room temperature for 10 minutes. It was then centrifuged at 300×g for 10 minutes. The supernatant was discarded and the cells were washed with 10 ml of PBS and centrifuged (10 minutes at 300×g) once again.

### Preparation of Total RNA

The RNeasy mini kit (QIAGEN, Hilden, Germany) was used, in accordance with the manufacturer's instructions, to prepare total RNA from the peritoneal cells. In this connection, two batches were in each case mixed for working up.

The yields of total RNA from the peritoneal cells derived from in each case four animals were between 10 and 20 μg of RNA.

### Intermediate Amplification of the RNA

A procedure based on the protocol of Eberwine et al. (1992) was used for carrying out the intermediate amplification of the RNA. This involves amplifying the mRNA from the preparation of total RNA.

### cDNA Arrant

The PIQOR™ cDNA array system from Memorec Stoffel GmbH (Cologne, Germany) was used for analyzing the induced and repressed genes. In connection with this, the cDNA derived from immunologically relevant genes (interleukins, differentiation clusters (CDs), transcription factors, receptors, etc.) was loaded onto a chip.

The amplified RNA was used for the hybridization. In each case 2 μg of the amplified RNA was transcribed into cDNA in an RT reaction and the fluorescence-labeled nucleotides were incorporated at the same time. The controls were labeled with Cy3 while the samples were labeled with Cy5.

The following hybridizations were carried out in accordance with the protocol specified by the manufacturer (PIQOR™ cDNA array system, edition 2.6, February 2000), and evaluated:

| Substance administered | Labels |
|---|---|
| H—LPEI, M_{w}: 87000, C18 acyl, 3 mol %, | Cy3: NaCl control |
| (0.1 mg per mouse) | Cy5: H—LPEI, M_{w}: 87000, |
| | C18 acyl, 3 mol % |
| H—LPEI, M_{w}: 87000, CDC acyl, 3 mol %, | Cy3: NaCl control |
| (0.1 mg per mouse) | Cy5: H—LPEI, M_{w}: 87000, |
| | CDC acyl, 3 mol % |

Only those signals in connection with which at least one of the samples (Cy3 and Cy5 signal, respectively) which were in each case to be compared exhibited a signal which was at least 2 times stronger than the mean value of the two negative controls (salt and herring sperm DNA) were analyzed when evaluating the hybridization signals. Only the signals of the genes which were expressed differentially more than two-fold were included for determining gene expression.

### Results

The following table provides a summary of the cDNA analysis. Values greater than 2.0 indicate a specific increase in the expression of the respective mRNA in (A), whereas values <−2.0 indicate suppression in (B). Following stimulation with the polyethyleneimines, it is in particular antiinflammatory or antiapoptotic factors which are overexpressed. The increase in the expression of interleukin-1 receptor type 2, to which the literature attributes a strongly antiinflammatory effect (Brown et al., 1996; Bossu et al., 1995), is extremely high. The expression of FERHA (ferritin heavy-chain mRNA) is described as being antiinflammatory or antiapoptotic (Weiss et al., 1997; Oberle & Schroder, 1997). The increase of MCL-1 to 2.88 also points to the polymers having an antiapoptotic effect (Fujise et al., 2000).

### Using the PIQOR™ cDNA Array System to Analyze the Effect of the Thyleneimines H-LPEI, M_{w}: 87000. C18 Acyl, 3 mol % and H-LPEI M_{w}: 87000, CDC Acyl, 3 mol % on Gene Expression in Murine Peritoneal Cells (In Vivo)

### A) Induced Genes

| | | H—LPEI, M_{w}: | H—LPEI, M_{w}: |
|---|---|---|---|
| | | 87000, C18 acyl, | 87000, CDC acyl, |
| No. | Name | 3 mol % | 3 mol % |
| 219 | CD121b/interleukin-1 receptor, type 2 | 21.99 | 47.92 |
| 201 | CD62/L-selectin | 4.45 | 4.13 |
| 119 | FERHA ferrithin heavy chain mRNA | 3.15 | 4.80 |
| 363 | lymphotoxin-beta | 3.13 | 3.51 |
| 139 | MCL1 | 2.87 | 2.88 |
| 204 | CD52 | 2.65 | 2.49 |
| 85 | CD128/high affinity interleukin 8 | 2.52 | 2.98 |
| | receptor A | | |
| 319 | CKR1 C—C CHEMOKINE RECEPTOR | 2.44 | 2.32 |
| | TYPE 1 (MIP-1 ALPHA-R)(RANTES- | | |
| | R) | | |
| 360 | BFL1 | 2.37 | 3.52 |
| 293 | interleukin 18 | 2.34 | −1.00 |
| 68 | SYCL-sysntenin | 2.32 | 2.06 |
| 296 | interleukin 15 | 2.27 | 1.23 |
| 294 | CD24 | 2.21 | 1.58 |
| 67 | Fgr tyrosine kinase Fgr (Src2) | 2.03 | 2.04 |
| 131 | GDF3 GROWTH/DIFFERENTIATION | 2.03 | 2.47 |
| | FACTOR 3 PRECURSOR | | |
| 152 | CD87/urokinase plasminogen activator | 1.95 | 2.37 |
| | surface receptor | | |
| 203 | GTPA GRPASE-ACTIVATING | 1.77 | 2.76 |
| | PROTEIN (RAS P21 PROTEIN | | |
| | ACTIVATOR) | | |
| 30 | Fas/CD95 | 1.53 | 2.69 |
| 234 | proteosome iota subunit (Glast) | 1.33 | 2.35 |
| 107 | PRDC | 1.32 | 2.00 |
| 377 | ENIGMA | 1.13 | 2.09 |
| 358 | FMLR FMET-LEU-PHE RECEPTOR | 1.06 | 3.82 |
| | (FMLP RECEPTOR) | | |
| 192 | AA2B ADENOSINE A2B RECEPTOR | 0.00 | 2.14 |

### B) Suppressed Genes

| | | H—LPEI, M_{w}: | H—LPEI, M_{w}: |
|---|---|---|---|
| | | 87000, C18 acyl, | 87000, CDC acyl, |
| No. | Name | 3 mol % | 3 mol % |
| 215 | TGF2 TRANSFORMING GROWTH | −10.72 | −9.06 |
| | FACTOR BETA 2 PRECURSOR | | |
| | (TGF-BETA 2) | | |
| 308 | EMR1 CELL SURFACE | −10.07 | −5.67 |
| | GLYCOPROTEIN EMR1 | | |
| | PRECURSOR | | |
| 390 | CD49f/integrin alpha-6 | −3.53 | −8.74 |
| 287 | CD29/integrin beta-1 | −3.46 | −1.81 |
| 109 | ETBR ENDOTHELIN B RECEPTOR | −3.04 | −2.70 |
| | PRECURSOR (ET-B) | | |
| 280 | RDC1 G PROTEIN_COUPLED | −2.87 | −2.48 |
| | RECEPTOR RDC1 HOMOLOG | | |
| 216 | CD136/macrophage-stimulating protein | −2.77 | −2.91 |
| | receptor | | |
| 297 | CD2 | −2.73 | −2.46 |
| 225 | CD115/macrophage colony stimulating | −2.67 | −1.49 |
| | factor I receptor | | |
| 285 | CD36 platelet glycoprotein IV | −2.58 | −2.54 |
| 57 | APRIL | −2.36 | −2.14 |
| 3 | actin | −2.35 | −1.89 |
| 61 | CD81/AAPA1 | −2.33 | −2.72 |
| 330 | CD147/Basigin | −2.26 | −2.03 |
| 347 | CD107a/LAMP1 | −2.13 | −1.62 |
| 257 | CD79a | −2.04 | −1.77 |
| 156 | GBAF GUANINE NUCLEOTIDE- | −1.75 | −2.05 |
| | BINDING PROTEIN G(OLF), ALPHA | | |
| | SUBUNIT | | |
| 376 | GAS3/PMP22 | 0.00 | −2.95 |
| 406 | P2Y5 P2Y PURINOCEPTOR 5 | 0.00 | −2.04 |
| | (PSY5) (PURINERGIC RECEPTOR 5) | | |

### LIST OF REFERENCES

Bossu, P., Visconti, U., Ruggiero, P., Macchia, G., Muda, M., Bertini, R., Bizzarri, C., Colagrande, A., Sabbatini, V. & Maurizi, G. (1995) Transfected type II interleukin-1 receptor impairs responsiveness of human keratinocytes to interleukin-1, Am.J.Pathol., 147, 1852-1861.

Brown, E. A., Dare, H. A., Marsh, C. B. & Wewers, M. D. (1996) The combination of endotoxin and dexamethasone induces type II interleukin 1 receptor (IL-1r II) in monocytes: a comparison to interleukin 1 beta (IL-1 beta) and interleukin 1 receptor antagonist (IL-1ra). Cytokine., 8, 828-836.

Eberwine, J., Yeh, H., Miyashiro, K., Cao, Y., Nair, S., Finnell, R., Zettel, M. & Coleman, P. (1992) Analysis of gene expression in single live neurons. Proc.Natl.Acad.Sci. U.S.A., 89, 3010-3014.

Elkins, K. L., Rhinehart-Jones, T. R., Stibitz, S., Conover, J. S. & Klinman, D. M. (1999) Bacterial DNA containing CpG motifs stimulates lymphocyte-dependent protection of mice against lethal infection with intracellular bacteria. J.Immunol., 162, 2291-2298.

Fujise, K., Zhang, D., Liu, J. & Yeh, E. T. (2000) Regulation of Apoptosis and Cell Cycle-Progression by MCL1. J.Biol.Chem., 275, 39458-39465.

Klinman, D. M., Verthelyi, D., Takeshita. F. & Ishii. K. J. (1999) Immune recognition of foreign DNA: a cure for bioterrorism? Immunity, 11, 123-129.

Krieg, A. M. (2000) The role of CpG motifs in innate immunity. Curr.Opin.Immunol., 12, 35-43.

Krieg, A. M., Love-Homan, L., Yi, A. K. & Harty, J. T. (1998) CpG DNA induces sustained IL-12 expression in vivo and resistance to Listeria monocytogenes challenge. J.Immunol., 161, 2428-2434.

Lucin, P., Jonjic, S., Messerle, M., Polic, B., Hengel, H. & Koszinowski, U. H. (1994) Late phase inhibition of murine cytomegalovirus replication by synergistic action of interferon-gamma and tumour necrosis factor. J. Gen. Virol., 75, 101-110.

March, J., Advanced Organic Chemistry, Wiley, N.Y., 4^{th} edition, 1992.

Oberle, S. & Schroder, H. (1997) Ferritin may mediate SIN-1-induced protection against oxidative stress. Nitric.Oxide., 1, 308-314.

Oxenius, A., Martinic, M. M., Hengartner, H. & Klenerman, P. (1999) CpG-containing oligonucleotides are efficient adjuvants for induction of protective antiviral immune responses with T-cell peptide vaccines. J. Virol., 73, 4120-4126.

Rivas, B. L., Ananias, S. I. (1992) Synthesis and characterization of poly(N-propionyl)iminoethylene. Polymer Bull., 28, 3-8.

Smith, P. M. Wolcott, R. M., Chervenak, R. & Jennings, S. R. (1994) Control of acute cutaneous herpes simplex virus. infection: T cell-mediated viral clearance is dependent upon interferon-gamma (IFN-gamma). Virology, 202, 76-88.

Steinmassl, M. & Wolf. G. (1990) Bildung der Zytokine Interleukin 2 und Interferon alpha durch mononukleäre Blutzellen des Schweines nach in vitro Stimulierung mit verschiedenen Viruspräparaten [Formation of zytokines interleukin 2 and interferon alpha by porcine mononuclear blood cells following in vitro stimulation with different virus preparations]. Zbl Vet Med, 37, 321-331.

Walker, P. S., Scharton-Kersten, T., Krieg, A. M., Love-Homan, L., Rowton, E. D., Udey, M. C. & Vogel, J. C. (1999) Immunostimulatory oligodeoxynucleotides promote protective immunity and provide systemic therapy for leishmaniasis via IL-12- and IFN-gamma-dependent mechanisms. Proc.Natl.Acad.Sci. U.S.A., 96, 6970-6975.

Walker, S., Sofia, M. J., Axelrod H. R. (1998) Chemistry and cellular aspects of cationic facial amphiphiles. Advanced Drug Delivery Reviews, 30, 61-71.

Weiss, G., Bogdan, C. & Hentze, M. W. (1997) Pathways for the regulation of macrophage iron metabolism by the anti-inflammatory cytokines IL-4 and IL-13. J.Immunol., 158, 420-425.

Zimmermann, S., Egeter, O., Hausmann, S., Lipford, G. B., Rocken, M., Wagner, H. & Heek, K. (1998) CpG oligodeoxynucleotides trigger protective and curative Th1 responses in lethal murine leishmaniasis. J.Immunol., 160, 3627-3630.

1. A medicament which comprises a polyamine as the active substance.

2. A medicament as claimed in claim 1, characterized in that the polyamine possesses hydrophobic substituents.

3. A medicament as claimed in claim 2, characterized in that the substituents are arranged as side chains or terminally.

4. A medicament as claimed in claim 2 or 3, characterized in that the substituents are alkyl chains, acyl chains or steroid-like substituents and also hydrophobic substituents which can be introduced by adding the nitrogen functions of the main polyamine chain to isocyanates or to α,β-unsaturated carbonyl compounds.

5. A medicament as claimed in one of claims 1 to 4, characterized in that the polyamine is a polyethyleneimine.

6. A medicament as claimed in claim 5, characterized in that the polyethyleneimine possesses the following general formula: 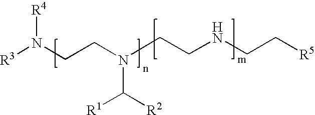
in which, in each individual [CH₂—CH₂—N] unit,
R¹ denotes hydrogen, methyl or ethyl, and
R² denotes alkyl having from 1 to 23 carbon atoms,
and in which
R³ and R⁴ (end groups) denote, independently of each other, hydrogen and alkyl having 1 to 24 carbon atoms, or possess a structure which is dependent on the initiator,
where R⁵ (end group) is a substituent which is dependent on the termination reaction,
and where the average degree of polymerization P=(m+n) is in the range from 45 to 5250 and n=a×P where 0.0001<a<0.1, with the units m and n being randomly distributed in the polymer.

7. A medicament as claimed in claim 5, characterized in that the polyethyleneimine possesses the following general formula: 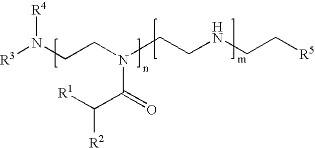
in which, in each individual [CH₂—CH₂—N] unit,
R¹ denotes hydrogen, methyl or ethyl, and
R² denotes alkyl having from 1 to 22 carbon atoms,
and in which
R³ and R⁴ (end groups) denote, independently of each other, hydrogen or acyl having from 1 to 24 carbon atoms, or possess a structure which is dependent on the initiator,
where R⁵ (end group) is a substituent which is dependent on the termination reaction,
and where the average degree of polymerization P=(m+n) is in the range from 45 to 5250 and n=a×P where 0.0001<a<0.1, with the units m and n being randomly distributed in the polymer.

8. A medicament as claimed in claim 5, characterized in that the polyethyleneimine possesses the following general formula: 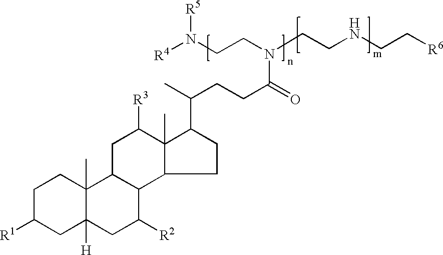
in which, in each individual [CH₂—CH₂—N] unit
R¹, R² and R³ denote hydrogen or hydroxyl,
and in which
R⁴ and R⁵ (end groups) denote, independently of each other, hydrogen or steroid parent substances, in particular bile acids, or possess a structure which is dependent on the initiator,
where R⁶ (end group) is a substituent which is dependent on the termination reaction,
and where the average degree of polymerization P=(m+n) is in the range from 45 to 5250 and n=a×P where 0.0001<a<0.1, with the units m and n being randomly distributed in the polymer.

9. A medicament as claimed in claim 5, characterized in that the polyethyleneimine possesses the following general formula: 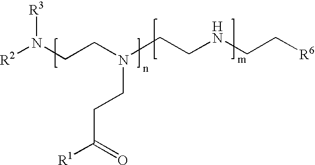
in which, in each individual [CH₂—CH₂—N] unit
R¹ denotes OR⁴ or NR⁴R⁵,
where
R⁴ and R⁵ denote, independently of each other, hydrogen or alkyl having from 1 to 24 carbon atoms,
and in which
R² and R³ (end groups) correspond, independently of each other, to the substituents of the nitrogen atoms of the main polymer chain or possess a structure which is dependent on the initiator,
where R⁶ (end group) is a substituent which is dependent on the termination reaction,
and where the average degree of polymerization P=(m+n) is in the range from 45 to 5250 and n=a×P, where 0.0001<a<0.1, with the units m and n being randomly distributed in the polymer.

10. A medicament as claimed in claim 5, characterized in that the polyethyleneimine possesses the following general formula: 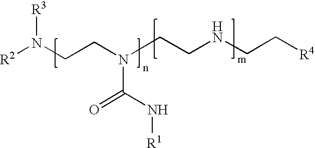
in which, in each individual [CH₂—CH₂—N] unit,
R¹ denotes alkyl having from 1 to 24 carbon atoms,
and in which
R² and R³ (end groups) correspond, independently of each other, to the substituents of the nitrogen atoms of the main polymer chain or possess a structure which is dependent on the initiator,
where R⁴ (end group) is a substituent which is dependent on the termination reaction,
and where the average degree of polymerization P=(m+n) is in the range from 45 to 5250 and n=a×P, where 0.0001<a<0.1, with the units m and n being randomly distributed in the polymer.

11. A medicament as claimed in claim 5, characterized in that the polyethyleneimine possesses the following general formula: 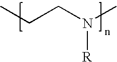
in which, in each individual [CH₂—CH₂—N) unit, the radical R can either be hydrogen or a radical of the formula 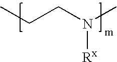
and in which R^{x} can either be hydrogen or also, once again, a radical of the type R,
and in which each individual [CH₂—CH₂—N] unit and the end groups can carry the substituents mentioned in claims 8 to 13,
and with the average degree of polymerization P=(m+n) being in the range from 45 to 5250.

12. A medicament as claimed in one of claims 1 to 11, characterized in that the polyamine has a molecular weight of less than 220000 g/Mol.

13. A medicament as claimed in claim 12, characterized in that the polyamine has a molecular weight of from 2000 to 100000 g/Mol.

14. A medicament as claimed in one of claims 1 to 13, characterized in that the polyamine is coupled to a cell-specific ligand.

15. A medicament as claimed in one of claims 1 to 14, characterized in that it additionally comprises formulation aids.

16. A polyamine as defined in claims 1 to 14 for use as a medicament.

17. The use of a polyamine as defined in claims 1 to 14 for producing an immunostimulatory medicament.

18. The use of a polyamine as defined in claims 1 to 14 for producing a medicament for the treatment of viral infections or for the prophylaxis of viral infections.

19. The use as claimed in claim 18, characterized in that the infections are infections with papilloma viruses, viruses of the herpes group, hepatitis viruses or HIV.

20. The use as claimed in claim 18, characterized in that the infections are infections of the respiratory tract or of the internal organs.

21. The use as claimed in claim 18, characterized in that the prophylaxis is the prevention of an infection due to stress or following an operation or dental treatment.

22. The use of a polyamine as defined in claims 1 to 14 for producing a medicament for the treatment of bacterial infections.

23. The use of a polyamine as defined in claims 1 to 14 for producing a medicament for the treatment of cancer or tumors

24. The use of a polyamine as defined in claims 1 to 14 for producing a medicament for the treatment of organ fibroses or for the prophylaxis of organ fibroses.

25. The use of a polyamine as defined in claims 1 to 14 for producing a medicament for the treatment of diseases which are accompanied by an increased deposition of collagen.

26. The use of a polyamine as defined in claims 1 to 14 for producing a medicament for the treatment of inflammatory, degenerative and proliferative diseases of the internal organs, the skin, the blood or the central nervous system and its appendages, including the eye.

27. The use of a polyamine as defined in claims 1 to 14 for producing a medicament for the treatment of diseases of the group of allergic diseases, in particular for the treatment of asthma.

28. The use of a polyamine as defined in claims 1 to 14 as an adjuvant.
